(19) United States
(12) Patent Application Publication
(10) Pub. No.: US 2021/0106628 A1
(43) Pub. Date: Apr. 15, 2021

(54) COMPOSITIONS AND METHODS FOR TREATING SKIN

(71) Applicant: RepliCel Life Sciences Inc., Vancouver (CA)

(72) Inventors: Rolf Hoffmann, Freiburg (DE); Kevin John McElwee, Vancouver (CA)

(21) Appl. No.: 16/912,565

(22) Filed: Jun. 25, 2020

Related U.S. Application Data
(63) Continuation of application No. 14/900,105, filed on Dec. 18, 2015, now abandoned.
(60) Provisional application No. 61/836,634, filed on Jun. 18, 2013.

(51) Int. Cl. A61K 35/36; C12N 5/071; A61K 35/16; A61K 45/06

(52) U.S. Cl. CPC A61K 35/36; C12N 5/0627; C12N 2533/80; A61K 45/06; A61K 35/16

(57) ABSTRACT

The present invention relates to compositions and methods utilizing hair follicle derived Non-Bulbar Dermal Sheath cells for use in the treatment of skin.

### CROSS-REFERENCE TO RELATED APPLICATION

This application is a continuation of U.S. application Ser. No. 14/900,105, filed Dec. 18, 2015, which was the National Stage of International Application No. PCT/US2014/043048, filed Jun. 18, 2014, which claims the benefit under 35 U.S.C. § 119(e) of U.S. Provisional Patent Application No. 61/836,634 filed Jun. 18, 2013, which applications are incorporated herein by reference in their entirety for all purposes.

### FIELD OF THE INVENTION

The present invention relates to compositions and methods for treating skin, as well for various cosmetic and aesthetic purposes, and more specifically, to compositions comprising of autologous or allogeneic non-bulbar dermal sheath (NDBS) cells for use in the treatment and repair of skin, including for example, skin injuries and for the prevention of skin aging, as well as for various cosmetic and aesthetic purposes.

### BACKGROUND

### Description of the Related Art

Skin is a complex organ and can be divided into at least three distinct layers, the epidermis, the dermis and the subcutis. Most of the fibrous connective tissue within the skin is formed by dermal fibroblasts, which secrete different types of collagens (such as type 1, 3 and others) to give the skin the firmness, to hold the epidermis and furthermore to secrete various autocrine and paracrine factors.

Skin can age or be injured in a number of ways, including for example, through external trauma (including surgical procedures), acne and other inflammatory reactions leading to scars, inherent diseases leading to fragile skin such as epidermolyis bullosa dystrophica with chronic wounds, chronic autoimmune inflammatory disease such as Scleroderma and variants, Borelliosis infection, Lupus erythematosis and variants, Lichen planus, the negative effect of smoking and UV-light (extrinsic aging) and general aging (intrinsic aging).

Skin aging is multifactorial and in addition to intrinsic/genetic mechanisms, smoking and UV-light has deleterious effects on the skin. Over the course of many years, dermal collagen degrades and thereby the contacts (e.g. integrins) of collagens to fibroblast diminish. As a consequence, aging fibroblasts show a rather rounded shape in contrast to the spindle shaped, multi-connected young fibroblasts. Over time this leads to skin laxity clinically visible as fine or sometimes more pronounce wrinkles on the face, the hands, the lower arms, the décolleté and elsewhere.

While there are a number of surgical and non-surgical methods that can be used to treat skin, but none of those techniques addresses the issue of a cellular deficit of functional fibroblasts in the aging skin.

The present invention discloses novel compositions and methods for treating skin aging and injuries, and further provides other related advantages.

### SUMMARY

Briefly stated, the present invention provides compositions and methods for treating or preventing skin aging or skin injuries utilizing hair follicle derived Non-Bulbar Dermal Sheath (“NDBS”) cells. Within one aspect of the present invention methods are provided comprising the steps of (a) preparing vital (i.e., ‘living’) hair; and (b) culturing the vital hair such that a population of NBDS cells can be obtained. Within preferred embodiments the NBDS cells are isolated.

Within one aspect of the invention methods are provided for isolating NBDS cells, comprising the steps of: (a) preparing vital hair; (b) cleaving the hair prepared in step (a) to remove the hair follicle bulb (which contains the dermal sheath cup and dermal papilla); (c) isolating Non-Bulbar Dermal Sheath tissue; and (d) cultivating the isolated Non-Bulbar Dermal sheath tissue to produce NBDS cells. Within one embodiment of the invention the vital hair is obtained by biopsy from the occipital scalp of a subject. Within another embodiment the hair is cleaved utilizing a micromanipulator and scalpel. Within yet other embodiments, the methods provided herein further comprise the step of conducting enzymatic digestion of the isolated Non-Bulbar Dermal Sheath tissue, optionally, with, for example collagen digesting enzymes such as collagenase, dispase, and leupeptin. Within further embodiments, the cells are passaged over multiple passages.

Within other aspects of the invention, isolated NBDS cells are provided, optionally prepared according to the methods described above, wherein the cells are isolated in order to provide a population which are primarily positive for one or more of: CD 90, CD73 and CD49b, and/or primarily negative for one or more of CD34, CD45 and KRT14 (optionally before or after culturing). Within preferred embodiments the isolated NBDS cells are at least 70%, 80%, 90%, 95%, 98% or 100% positive for one or more of the positive markers described above, and/or at least 80%, 90%, 95%, or 98% negative for one of the negative markers described above.

Within preferred embodiments of the invention, isolated NBDS cells have less than 15%, 10%, 5%, or 1% keratinocytes within the cell population and/or less than 15%, 10%, 5%, or 1% melanocytes within the cell population. However, within further embodiments, the isolated NBDS cell population is derived from a population of dermal cells (preferably from a hair follicle) that have some contaminating cell types, including for example, at least 1, 5, 10, 0.01%, 0.1%, or 1% keratinocytes in the cell population, and/or at least 5, 10, 0.1%, 0.01% melanocytes. Within further embodiments of the invention the isolated NBDS cells are at least 95% pure, and have at least one contaminating cell type (e.g., at least one keratinocyte) within the cell population.

These NBDS cells (or expanded or isolated NBDS cells) may be contained within compositions with other ingredients, such as, for example, serum plasma, fibrin, and/or hyaluronic acid. Within other embodiments the NBDS cells (or expanded or isolated NBDS cells) may be constituted in a composition suitable for injection, e.g., Lactated Ringer's or a buffered saline solution. Other ingredients which may be utilized to form the compositions of the present invention include, for example, components of the extracellular matrix (e.g., glycosaminoglycans (GAGs), heparin sulfate, chondroitin sulfate, keratin sulfate, hyaluronic acid, albumin (e.g., human albumin), elastin, fibronectins and laminins), cytokines and chemokines (e.g., transforming growth factor beta (TGF-beta) and its isoforms, insulin-like growth factor (IGF) and its isoforms, granulocyte-macrophage colony-stimulating factor (GM-CSF), parathyroid-hormone-related protein, hepatocyte growth factor/scatter factor (HGF/SF), macrophage stimulating protein (MSP), epidermal growth factor (EGF), interleukin 6 (IL-6), stromal cell-derived factor 1 (SDF-1), platelet derived growth factor (PDGF) and fibroblast growth factor (FGF) and/or various therapeutic agents (e.g., analgesic agents, anti-inflammatory agents and immunomodulatory agents). Within other embodiments however NBDS cells (and isolated NBDS cells) are provided in compositions that do not have any of the aforementioned ingredients (including for example, serum or plasma).

Within yet other aspects of the invention methods are provided for treating the skin of a subject, comprising the step of administering to the skin of a subject a composition comprising NBDS cells as described herein. Within one embodiment, the subject is a mammal selected from the group consisting of humans, horse, dogs and cats. Within various embodiments the treatment is due to a skin injury. Within certain embodiments, the skin injury can result from external trauma (e.g., a surgical procedure or wound, burn, radiation, or an accident), or an acute or chronic wound or scar. Within other embodiments the skin injury is a predisposition of spontaneous or induced bruises on the extremities. Within yet other embodiments the skin injury is due to acne and/or other inflammatory reactions leading to scars, inherent disease with fragile skin such as epidermolyis bullosa dystrophica or other inherent or acquired blistering diseases with chronic wounds incurred by diabetes mellitus, arteriosclerosis or varicosis, acute or chronic viral, mycotic or bacterial infections, chronic autoimmune inflammatory disease such as Scleroderma and variants, Borelliosis infection, Lupus erythematosis and variants, Lichen planus, the negative effect of smoking and UV-light (extrinsic aging) and general aging (intrinsic aging). Within yet another embodiment, the skin is on the entire human body, or a selected portion of the body (such as the hands, face, and neck. Within other embodiments, the skin can be aged/damaged by sun (UV-light) or smoking. Within related embodiments the aged skin suffers from a conditions selected from the group consisting of fine or coarse wrinkles, decreased skin thickness, decrease elasticity and firmness. Within other embodiments the skin is treated for cosmetic purposes (e.g., to reduce the appearance of cellulite). Within yet other embodiments, there is no current visible injury or trauma to the skin, but the skin may nevertheless be treated in order to prevent or delay the incidence of normal aging, or to improve the appearance (and texture) of normal skin.

Within another aspect of the invention compositions are provided comprising NBDS cell culture supernatants. Within one embodiment, the NBDS cell culture supernatant is prepared from cultured NBDS cells as described herein. Within other embodiment the NBDS cell culture supernatant can be stored by freezing, lyophilisation, or other suitable methods. The NBDS cell culture supernatant can be utilized in a variety of methods, including for example, applied topically, injected (e.g., small injections to the skin or other body parts).

Within various embodiments, the NBDS cell culture supernatant can be prepared and utilized as is (e.g., after filtration), diluted with suitable buffers or excipients, concentrated, or with various components separated out (which may be utilized separately (e.g., stem cell factor, wnt-factors, other growth factor, cytokines or chemokines). Within one embodiment the NDBS cell culture supernatant is utilized as is, diluted or concentrated, and combined or admixed with a compound or compositions suitable for administration (e.g., a gel, creme, or polymer-based composition). Representative examples include hyaluronic acid, collagen, extracellular matrix, scaffolds, polymer-based compositions (e.g., polyethylene glycols, poly-lactic acids, and poly caprolactones), Other suitable carriers for the NBDS cell culture supernatants include pastes, and other liquids utilized in the cosmetic industry (e.g., emulsions, laquers, tonics, shampoos, sprays). Within other embodiments, the NDBS cell culture supernatants may be placed in manufactured or harvested extracellular matrices (see e.g. US 20100047305 or US 20100124573). Within other embodiments, the cells may be utilized on a medical device, or placed on or within a non-biodegradable, or, biodegradable scaffold, or other structure. Particularly preferred scaffolds or structures include biodegradable scaffolds (e.g., collagen- based scaffolds, such as, for example, meshes). Representative examples of suitable scaffolds include, for example, U.S. Pat. Nos., 5,736,372, 5,759,830, 8,039,258 and 8,105,380, all of which are incorporated by reference in their entirety.

The details of one or more embodiments are set forth in the description below. Other features, objects and advantages will be apparent from the description, the drawings, and the claims. In addition, the disclosures of all patents and patent applications referenced herein are incorporated by reference in their entirety.

### BRIEF DESCRIPTION OF THE DRAWINGS

FIGS. 1A, 1B, 1C, and 1D illustrate the dissection of a human hair follicle. FIG. 1A shows an isolated human hair follicle, which can be cleaved above the bulbar portion of the hair root (i.e., above the dermal papillae and dermal sheath cup cells, i.e., above the end bulbs), but below the base of the sebaceous gland canal, in order to obtain an isolated dermal sheath (see FIG. 1B). The structure depicted in FIG. 1B can be separated into at least two separate components, as shown in FIGS. 1C and 1D. FIG. 1C depicts the hair fiber and associated inner root sheath, and outer root sheath which contain predominantly keratinocytes, and FIG. 1D is the dermal sheath containing NBDS cells (also occasionally referred to as the connective-tissue sheath, upper dermal sheath or less precisely just dermal sheath). In contrast to dermal sheath cup cells (DSC) and dermal papilla (DP) cells, NBDS cells are highly positive for a collagen-1 marker and only weakly for alkaline phosphatase and steroidsulfatase. In addition, these cells express markers such as CD90 and other stem cell markers.

FIG. 2 is an illustration of a hair follicle depicting the origin for dermal papillae (“DP”) cells, dermal sheath cup (“DSC”) cells, and Non-Bulbar Dermal Sheath (“NBDS”) cells.

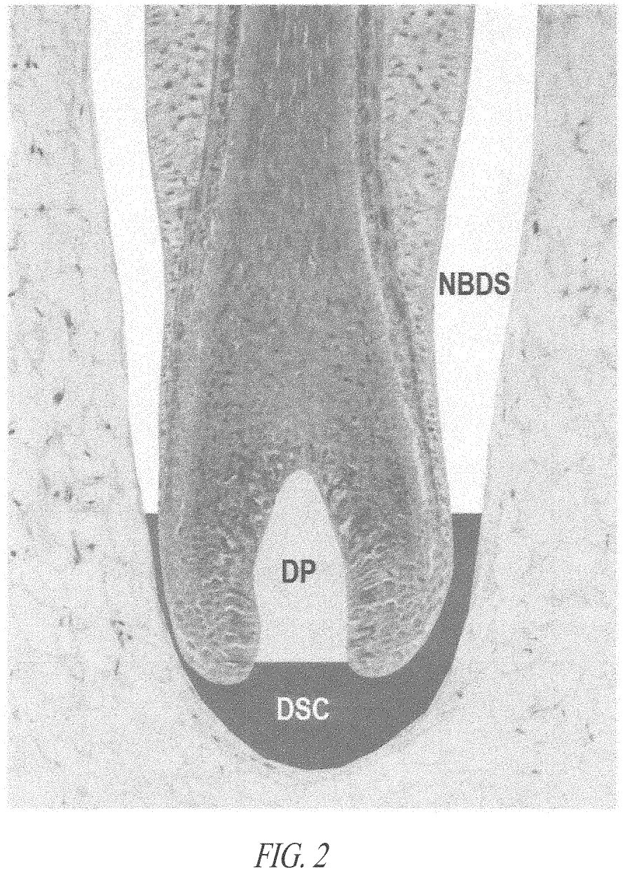

FIG. 3 is a photomicrograph of NBDS cells in culture.

### DETAILED DESCRIPTION OF THE INVENTION

As noted above, the present invention provides hair follicle derived Non-Bulbar Dermal Sheath (NDBS) cells for use in the treatment or prevention of skin aging and skin injuries within a mammal. Prior to setting forth the invention however, it may be helpful to an understanding thereof to first set forth definitions of certain terms that are used hereinafter.

Non-Bulbar Dermal Sheath cells, or “NBDS” cells, refers to dermally derived cells (or more specifically, derived from hair follicles). Within preferred embodiments, the sheath cells are obtained from the outer dermal sheath of a hair follicle, above the bulbar portion of the hair root (i.e., above the dermal papilla and dermal sheath cup cells), but below the base of the sebaceous gland canal. Within particularly preferred embodiments of the invention NBDS cells are UV-naive cells, i.e., they have not been exposed to substantive UV exposure (for example, as compared to the nose or the top of your hands). NBDS cells can be expanded and isolated as described in more detail below.

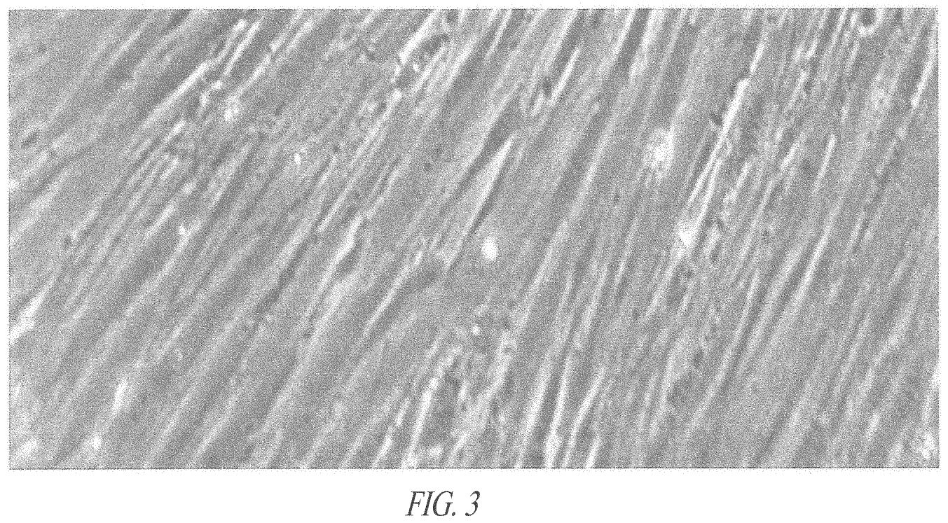

NBDS cells may be readily identified by a number of methods, including for example, by the method of preparation and culture (as described below); morphology (see, e.g., FIG. 3); as well as cell specific markers (e.g., NBDS cells are primarily positive for CD 90, CD73 and CD49b, and/or primarily negative for CD34, CD45 and KRT14, either before or after culturing). In all events however, the cells must be of a dermal origin, or, within more preferred embodiments, from a hair follicle.

Expanded Non-Bulbar Dermal Sheath cells, or “eNBDS cells” refers to NBDS cells which have been expanded for several passages in culture, but which retain the ability to produce collagen (e.g., type I collagen) as well as a variety of cytokines, chemokines and hormones. As above, unexpectedly, the eNBDS cells can also be immunoregulatory. Within preferred embodiments, the cells can be expanded in culture for 1, 2, 3, 4, 5, 10, 20 or more passages.

“Isolated” NBDS cells refers to a cell population of greater than 70%, 80%, 85%, 90%, 95%, 98%, or 100% NBDS cells. NBDS cells have the ability to produce collagen (e.g., type I collagen), as well as a variety of cytokines and chemokines. Unexpectedly, the NBDS cells can also be immunoregulatory, making them particularly suitable for treatment of skin injuries (e.g., by assisting in suppressing any inflammatory response).

Within certain embodiments of the invention, software or other visualization techniques that can be utilized to visualize cells on a microscopic scale can be used to assess the size, shape, viability and granularity of a large number of cells in a visual field, and ascertain the number of NBDS cells (which are fibroblast-like—as shown in FIG. 3), as opposed to keratinocytes, melanocytes DSCs, and other cell types which are of different morphology). Hence, within one embodiment of the invention methods are provided for isolating NBDS cells comprising the step of culturing cells over at least 1, 2, 3, 4, 5, 6, 10, or 20 passages from a hair follicle such that an isolated population of NBDS cells is produced. Within preferred embodiments the cells placed into dishes or flasks which allow the NBDS cells to adhere, and with each passage non-adherent cells are removed, and the remaining adherent cells released (e.g., by trypsinization), followed by addition of fresh media. Within such embodiments it can be determined when a sufficient population of isolated NBDS cells has been obtained by visualizing the cells in the cell culture in order to assess the number of NBDS cells vs. non-NBDS cells. Visualization techniques include, but are not limited to direct microscopic visualization, staining of the cells for markers (or lack thereof—e.g., for lack of keratin), and light/laser analysis to look at diffraction patterns of the different cell types (see, generally “Laser Scanning Microscopy and Quantitative Image Analysis of Neuronal Tissue” Lidia Bakota and Roland Brandt, eds., Humana Press, 2014; see also “Imaging and Spectroscopic Analysis of Living Cells: Optical and Spectroscopic Techniques”, Conn ed., Academic Press, 2012)

Within other embodiments, cell specific markers (e.g., NBDS cells are primarily positive for CD 90, CD73 and CD49b, and/or primarily negative for CD34, CD45 and KRT14 (optionally before or after culturing) can be utilized to assess the degree of NBDS cells vs. contaminant cell types. “Applications of Flow Cytometry in Stem Cell Research and Tissue Regeneration”, Krishan, Krishnamurthy, and Totey eds., Wiley-Blackwell, 2010). For example, isolated NBDS cells may be prepared by a) obtaining one or more vital hair follicles; b) releasing cells from the hair follicle (e.g., through the use of enzymes, or by culturing growing cells out of the hair follicle); and c) sorting the cells (e.g., by flow cytometry or through the use of magnetic beads) to obtain a population of isolated NBDS cells. Within certain embodiments of the invention cells in any stage of the process may be optionally cultured as described above (e.g., cells may be cultured for at least 1, 2, 3, 4, 5, 6, 10 or 20 passages as described above, and the resultant cells further isolated by, for example, flow cytometry or magnetic beads.

Within preferred embodiments the isolated NBDS cells are at least 70%, 80%, 85%, 90%, 95%, 98%, or 100% positive for one or more of the positive markers described above, and/or at least 80%, 90%, 95%, or 98% negative for one of the negative markers described above.

Within further preferred embodiments of the invention, the NBDS cells are isolated from ultraviolet naïve tissue, and in particular, from follicles that are covered by hair, and hence, have less ultraviolet exposure and/or injury than, for example, dermal cells on the hands or forearms.

Within preferred embodiments of the invention (and utilizing any of the techniques described herein), isolated NBDS cells have less than 15%, 10%, 5%, or 1% keratinocytes within the cell population and/or less than 15%, 10%, 5%, or 1% melanocytes within the cell population. However, within further embodiments, the isolated NBDS cell population is derived from a population of dermal cells (preferably, from hair follicles) that have some contaminating cell types, including for example, at least 1, 5, 10, 0.0.01%, 0.1%, or 1% keratinocytes in the cell population, and/or at least 5, 10, 0.1%, 0.01% melanocytes. Within further embodiments of the invention the isolated NBDS cells are at least 95% pure, and have at least one contaminating cell type (e.g., at least one keratinocyte) within the cell population.

“Skin injuries” refers to damage of the skin due to external or internal trauma. For example, skin injuries may be caused by acne and/or other inflammatory reactions leading to scars or wounds, inherent diseases with fragile skin or blistering diseases such as epidermolyis bullosa dystrophica with chronic wounds, chronic autoimmune inflammatory diseases such as scleroderma and variants, Borelliosis and other infections, Lupus erythematosis and variants, Lichen planus, Psoriasis, atopic dermatitis, pruritus. Skin injuries may also be due to surgical procedures or wounds, and accidents. Wounds may be caused by a number of factors, including diabetes and venous or arterial insufficiency. Skin injuries can also be caused by a number of factors, including burns, the cold, radiation, or, induced by various medications. Skin injuries can also be due to “skin aging,”, which refers to the negative effect of various factors, including for example, smoking, UV-light (extrinsic aging) and general aging (intrinsic aging)

### Preparation of NBDS Cells

As noted above, the present invention provides methods for isolating NBDS cells. Within one aspect of the present invention such methods comprise the steps of (a) preparing vital hair; and (b) culturing the vital hair such that a population of NBDS cells can be obtained. With respect to step (a), a wide variety of methods may be utilized to obtain vital hair, including for example, surgical methods to remove a variety of hair follicles (along with the skin), or by plucking one or more hair follicles directly from a subject.

Once the vital hair has been obtained, it can be cultured under conditions which allow, and preferentially, promote the growth of NBDS cells. Within preferred embodiments, this culturing under conditions wherein fibroblast-like cells are allowed to proliferate. Within preferred embodiments the step of culturing is performed with serum-free media. After several passages (e.g., at least 2, 3, 4, 5, 10 or more passages), the cultured cells are analysed as described above in order to ascertain whether there is a sufficient quantity of NBDS cells, and whether the cells have been sufficiently isolated from contaminating cells.

Within other aspects of the invention, methods are provided comprising the steps of (a) preparing vital hair; (b) cleaving the hair prepared in step (a) to remove the hair follicle bulb (which contains the dermal sheath cup and dermal papilla); (c) isolating Non-Bulbar Dermal Sheath tissue; and (d) cultivating the isolated Non-Bulbar Dermal sheath tissue to produce NBDS cells.

In order to prepare vital (or ‘living’) hair, a sample is typically obtained from a given subject (e.g., a mammal such as a human, horse, pig, cat, dog, rabbit, guinea pig, rat or mouse). The sample may be obtained from a variety of sites (e.g., for humans, from the occipital area of the scalp, the chest or thigh, and for horses from the mane or tail). The sample may be obtained via a biopsy, or other suitable means (e.g., by ‘plucking’, or dissection). Preferably, hair follicles in the anagen phase of development are selected, although other phases of development (e.g., the catagen phase) can also be utilized.

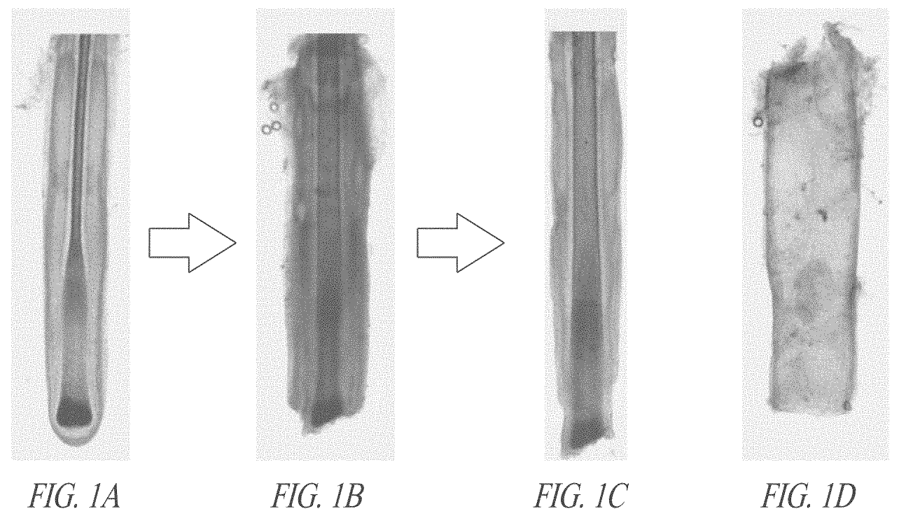

Once the sample is obtained from the subject, the sample is then separated to isolate the hair follicle, typically utilizing a micromanipulator and scalpel, although other instruments such as needles may also be utilized. Within certain embodiments, the isolated hair follicle as shown in FIG. 1A can be further cleaved above the bulbar portion of the hair root (i.e., above the dermal papillae and dermal sheath cup cells), but below the base of the sebaceous gland canal in order to obtain an isolated dermal sheath (see FIG. 1B). The structure depicted in FIG. 1B can be separated into at least two separate components, as shown in FIGS. 1C and 1D. FIG. 1C depicts the hair fiber and associated inner root sheath, and outer root sheath which contain predominantly keratinocytes, and FIG. 1D is the dermal sheath containing NBDS cells (also occasionally referred to as the connective-tissue sheath).

The dermal sheath (FIG. 1D) can, within certain embodiments, be further separated, for example, by cutting length-wise along one side, or, by using techniques such as enzymatic digestion (e.g., with collagen digesting enzymes such as collagenase, dispase and leupeptin).

The dermal sheath containing NBDS cells, or the separated NBDS cells can then be cultured in a medium (either with or without serum) which promotes cell proliferation (see e.g., FIG. 3). Suitable media include, for example, DMEM/Hams F12 supplemented with fibroblast growth factor (FGF), fetal calf/bovine serum and antibiotics. Alternatively, cells can be replicated in a serum-free process, in which various combinations of serum-free media and supplements are utilized. The examples of serum-free media include X-Vivo™ and TheraPEAK™ FGM-CD™ containing serum supplements and/or human derived platelet extract. After 3 to 5 days, fresh proliferation medium is typically added to the culture medium. Subsequently the medium can be changed every 2 to 4 days. When the culture has reached approximately 80 to 90% confluence, the cells are detached from the culture flask via, for example, trypsinization, and seeded in a larger tissue culture flask. This step is repeated for a number of passages (e.g., 2, 4, or 6) until approximately 5 to 100 million cells are obtained.

Within certain embodiments of the invention, the cells may be cultured under conditions of low oxygen tension (for example, as described in U.S. Patent Pub. No. US 2013/0177537, which is incorporated by reference in its entirety).

Once the desired number of cells are obtained, the cells are washed several times, trypsinized, and resuspended in cell transportation medium (CTM), which is composed of ringer lactate, 10-20% human serum albumin (HAS) and 2-5% dimethylsulfoxide (DMSO). Cells are counted and adjusted to provide the final concentration of 20 million cells/ml and stored in liquid nitrogen.

All cell culture supernatants will not be discarded as they contain individual growth factors, matrix molecules, stem cell factors, made by the patients' cells. Cell culture supernatants will be frozen, freeze-dryed or any other storage method to be suitable for the specific use.

### Preparation of Compositions Comprising NBDS Cells

As noted above, NBDS cells may be contained within compositions with other ingredients, such as, for example, blood serum or plasma, platelet-rich plasma (PRP), fibrin, and/or hyaluronic acid. Other commercially available products may also be utilized to prepare suitable compositions, including for example, TISSEEL and COSEAL (available from Baxter), TISSUCOL, BERIPLAST, QUIXIL, TACHOSIL, and EVICEL. Other polymer-based compositions may also be utilized, including for example, polyethylene glycols, poly-lactic acids, and poly caprolactones. Within other embodiments, the cells may be place is either manufactured or harvested extracellular matrices (see e.g. US20100047305 or US20100124573). Within other embodiments, the cells may be placed within a non-biodegradable, or, biodegradable scaffold, or other structure. Particularly preferred scaffolds or structures include biodegradable scaffolds (e.g., collagen- based scaffolds, such as, for example, meshes). Representative examples of suitable scaffolds include, for example, U.S. Pat. Nos., 5,736,372, 5,759,830, 8,039,258 and 8,105,380, all of which are incorporated by reference in their entirety.

Within other preferred embodiments the composition is provided in one or two or more parts (e.g., in a double barrelled syringe that admixes components, or in bi- or multichambered cartridge) that is freely flowing and injectable. Representative examples of such syringes include those described in U.S. Pat. Nos. 5,750,657 and 8,039,021, which are both incorporated by reference in their entirety.

Other ingredients may also be included within these compositions, including for example, components of the extracellular matrix (e.g., glycosaminoglycans (GAGs), heparin sulfate, chondroitin sulfate, keratin sulfate, hyaluronic acid, elastin, collagens, fibronectins and laminins), cytokines and chemokines (e.g., transforming growth factor beta (TGF-beta) and its isoforms, insulin-like growth factor (IGF) and its isoforms, granulocyte-macrophage colony-stimulating factor (GM-CSF), parathyroid-hormone-related protein, hepatocyte growth factor/scatter factor (HGF/SF), macrophage stimulating protein (MSP), epidermal growth factor (EGF), interleukin 6 (IL-6), stem cell factor (SCF) stromal cell-derived factor 1 (SDF-1), platelet derived growth factor (PDGF) and fibroblast growth factors (FGF) and/or various therapeutic agents (e.g., analgesic agents, anti-inflammatory agents, antibiotics, antimycotics, antiviral and immunomodulatory agents).

### Methods for Treating Skin Utilizing NBDS Cells

Methods are also provided for treating or preventing skin aging or skin injuries, comprising the step of administering to a subject a composition comprising NBDS cells are described above. Typically, cells are administered by injection, although within various embodiments, to the extent a surgical method is employed the cells may be provided directly into, beside or underneath an open wound.

Fibroblasts are the main cell type in the skin dermis. They are highly biologically active and for a multi-layered, 3-dimensional network of cells, intermingled with collagen and connected via surface receptors such as integrins. There is a constant renewal and breakdown of collagen fibres by enzymes such an MMP's (matrix metalloproteinases) which regulate the volume and tightness of the skin. With aging, UV-exposure or smoking collagen degrades and cannot be replaced by the aged fibroblast. The fibroblasts become rather inactive, more round in appearance and produce less collagen. This leads to a thinner skin with less tensile strength. Eventually this leads to an aged skin with small wrinkle lines but also larger wrinkles as seen in the nasolabial fold, the forehead, the cheeks and elsewhere.

Thus, as noted above, the present disclosure provides compositions and methods for treating the skin of a subject, comprising the step of administering to the skin of a subject a composition comprising NBDS cells as described herein. Within one embodiment, the subject is a mammal selected from the group consisting of humans, horse, dogs and cats. Within various embodiments the treatment is due to a skin injury. Within certain embodiments, the skin injury can result from external trauma (e.g., a surgical procedure or wound, burn, radiation, or an accident), or an acute or chronic wound or scar. Within other embodiments the skin injury is a predisposition of spontaneous or induced bruises on the extremities. Within yet other embodiments the skin injury is due to acne and/or other inflammatory reactions leading to scars, inherent disease with fragile skin such as epidermolyis bullosa dystrophica or other inherent or acquired blistering diseases with chronic wounds incurred by diabetes mellitus, arteriosclerosis or varicosis, acute or chronic viral, mycotic or bacterial infections, chronic autoimmune inflammatory disease such as Scleroderma and variants, Borelliosis infection, Lupus erythematosis and variants, Lichen planus, the negative effect of smoking and UV-light (extrinsic aging) and general aging (intrinsic aging). Within yet another embodiment, the skin is on the entire human body, or a selected portion of the body (such as the hands, face, and neck. Within other embodiments, the skin can be aged/damaged by sun (UV-light) or smoking. Within related embodiments the aged skin suffers from a conditions selected from the group consisting of fine or coarse wrinkles, decreased skin thickness, decrease elasticity and firmness. Within other embodiments the skin is treated for cosmetic purposes (e.g., to reduce the appearance of cellulite). Within yet other embodiments, there is no current visible injury or trauma to the skin, but the skin may nevertheless be treated in order to prevent or delay the incidence of normal aging, or to improve the appearance (and texture) of normal skin.

Within various embodiments of the invention NBDS cells may be administered by way of a device such as that disclosed within PCT Publication No. WO 2013/113121, which is incorporated by reference in its entirety.

A large number of species may be treated with NBDS cells and compositions provided herein, including for example, mammals such as humans, horse, dogs and cats.

Cosmetic/Aesthetic Procedures Utilizing NBDS Cells or Cell Culture Supernatants of thereof

As discussed above, a variety of signs of skin aging can be readily treated and/or prevented via intracutaneous (i.c), intradermal or subcutaneous (s.c.) injection of NDBS cells. Within other embodiments, the skin of a subject may be treated with a composition comprising NBDS cell culture supernatants. Within various embodiments of the invention the entire skin of a subject (e.g., a human subject) can be treated, although, within but more specific aspects, cosmetic and aesthetic treatments can be provided to the subject. Representative examples of such treatments include, but are not limited to: fine and coarse wrinkles at the cheeks, the nose, the ears, the forehead, the nasolabial fold, the eye lids, around the eyes (crow's feet), around the lips, the chin, the temples, the neck, the décolleté, the breast, the trunk, the hands and arms, the legs and feet. Essentially all parts of the skin which may be prone to UV-damage can benefit from such a treatment. In addition those body parts suffering from loss of volume can be treated by the injection of NBDS cells as this will increase volumes by adding cells and thereby collagen, or by use of compositions comprising NBDS cell culture supernatants. Therefore tissue augmentation of the ear, the ear lobes, the nose, the lips, the cheeks, the neck, the décolleté, the breasts, the nipples, the genitals are another embodiments.

Within various embodiments, the NBDS cells can be delivered via injections, which can be done with or without local or systemic analgesia or sedation. This can be done with a single or a multi-needle device. In addition, it can be performed either by a single injection as a bolus or multiple, multilayered injections with different techniques such as criss-cross, feathering or others. The delivered volumes/cell numbers large depend of the indication and the area to be treated. Typical doses may start from as low as 0.01m1 up to several ml. In certain aspects of the invention the injected cell numbers may range from 10 to billions of cells, and more preferably, from 100, 1,000, 10,000, 100,000, 1,000,000 and/or 10,000,000 up to a billion or more cells. The number of injected cells, will depend on, among other things, the size of the area to be treated, the total number of cells available and the volume injected, as well as the desired degree of efficacy.

In addition, injection of NDBS cells into, around, or underneath an acute or chronic wound will be beneficial for wound healing. Wounds may arise from those disease defined herein.

Within yet other aspect of the invention NDBS cell culture supernatants are provided in a suitable cosmetic preparation. Specifically, cell culture supernatant can be applied to the skin by itself, or concentrated and admixed with other ingredients suitable for use as a cosmetic preparation. Suitable excipients are described in, for example, U.S. Pat. Nos. 8,343,466, 8,343,520, and 8,349,338, and in “A Formulary of Cosmetic Preparations”, 1977, by Michael Ash, all of which are incorporated by reference in their entirety.

Compositions comprising such NBDS cell culture supernatants can be utilized to treat both normal skin, as well as a variety of skin injuries. As discussed in more detail above, a skin injury can result from external trauma (e.g., a surgical procedure or wound, burn, radiation, or an accident), or an acute or chronic wound or scar. Within other embodiments the skin injury is a predisposition of spontaneous or induced bruises on the extremities. Within yet other embodiments the skin injury is due to acne and/or other inflammatory reactions leading to scars, inherent disease with fragile skin such as epidermolyis bullosa dystrophica or other inherent or acquired blistering diseases with chronic wounds incurred by diabetes mellitus, arteriosclerosis or varicosis, acute or chronic viral, mycotic or bacterial infections, chronic autoimmune inflammatory disease such as Scleroderma and variants, Borelliosis infection, Lupus erythematosis and variants, Lichen planus, the negative effect of smoking and UV-light (extrinsic aging) and general aging (intrinsic aging). Within yet another embodiment, the skin is on the entire human body, or a selected portion of the body (such as the hands, face, and neck. Within other embodiments, the skin can be aged/damaged by sun (UV-light) or smoking. Within related embodiments the aged skin suffers from a conditions selected from the group consisting of fine or coarse wrinkles, decreased skin thickness, decrease elasticity and firmness. Within other embodiments the skin is treated for cosmetic purposes (e.g., to reduce the appearance of cellulite). Within yet other embodiments, there is no current visible injury or trauma to the skin, but the skin may nevertheless be treated in order to prevent or delay the incidence of normal aging, or to improve the appearance (and texture) of normal skin.

The following examples illustrate the invention and should not be understood as limiting the scope of the invention.

### EXAMPLE 1

### Tissue Sampling

A skin biopsy from the occipital area of the scalp is obtained from a subject as follows. Briefly, once an appropriate area of the scalp has been selected, it is shaved with hair clippers, ensuring some stubble remains. The biopsy area is then thoroughly disinfected and anaesthetized. Once anesthesia has taken effect, a 1-10 mm deep punch or an excisional biopsy is gently removed from the biopsy site and the incision closed with sutures which can be removed 8-16 days later. The skin biopsy is then packaged under aseptic conditions into a pre-labelled biopsy tube containing transport medium.

### EXAMPLE 2

### Isolation and Cultivation of NBDS Cells

A sterility test is performed on the medium in which the biopsy has been transported to ensure the sample is free from contamination, or alternatively, if the sample is contaminated to ensure that medium with antibiotics is subsequently utilized. The biopsy is then washed several times to remove the biopsy transportation medium and any debris to prepare the tissue for subsequent processing. Hair follicles are processed in Hams F10 by cutting away the skin epithelium with a sterile scalpel and “plucking” or dissecting the whole hair follicle unit from the surrounding dermal tissue using sterile forceps. The hair follicle is gripped with a forceps as close as possible to the skin surface and the follicle exposed by pulling up on the hair in the hair follicle unit. Follicles in the anagen phase (growing phase of the hair cycle, indicated by the visible outer root sheath, and DSC of the hair bulb) are selected for further processing.

NBDS isolation is performed in Hams F10 by first detaching the follicular dermal sheath cup cells and papilla from the rest of the hair follicle using a fine sterile mini-scalpel or needle, and discarded. The dermal sheath containing NBDS cells is removed, and the tissue is prepared for cultivation.

Six to ten dermal sheath tissues are gently placed into 3% hyaluronic acid gel and covered with cell proliferation promoting culture medium such as, for example, DMEM/Hams F12 supplemented with FGF, 10% FCS and antibiotics. After 3 to 5 days, fresh proliferation medium is added to the culture. Subsequently the medium is changed every 2 to 4 days. When the culture has reached approximately 80 to 90% confluence, the cells are detached from the culture flask via trypsinization, and seeded in larger tissue culture flasks. This step is repeated for four passages to obtain approximately 100 million cells.

Once approximately 100 million cells are obtained, the cells are washed with PBS, trypsinized and resuspended in Cell Transportation Medium (CTM: Ringer lactate containing 10% human serum albumin and 5% dimethylsulfoxide). The cells are sedimented by centrifugation and pooled together. The supernatant is aspirated and the cell pellet is resuspended in CTM. Two cell samples/aliquots are removed from the cell-CTM mixture for quality control and cell counting. After the cells are counted, they are sedimented once more by centrifugation, and the resulting pellet is resuspended in CTM to give a final concentration of 20 million cells/ml. The final cell products are stored below −130° C. in liquid nitrogen till shipment.

### EXAMPLE 3

### Preparation and Administration of NBDS Cells into Skin

Skin on the face is first prepared for injection by application of a topical analgesia (e.g., EMLA-cream) for approximately one hour. Thereafter, the skin is washed and disinfected. NMDS cells, prepared as described above, are then injected into the skin in a repetitive manner, in order to cover the entire surface of the desired treatment area. The skin may be cooled with ice as necessary.

### EXAMPLE 4

### Use of Cell Culture Supernatants in Cosmetic Preparation

As an adjunct, a follow-up or as a stand alone treatment, cell culture supernatants can be used to have a beneficial effect on skin structure, texture, look, moisture, thickness and firmness.

Briefly, cell culture supernatants taken from the growing NBDS cells as described in Example 2 can be used alone, concentrated or dissolved in typical cosmetic excipients and applied to aged or injured skin.

The present disclosure provides the following specific embodiments, which are not limiting on the invention, but are exemplary of the embodiments disclosed herein.

1) A method for isolating NBDS cells, comprising:

(a) preparing vital hair;

(b) cleaving the hair prepared in step (a) to remove the hair follicle bulb;

(c) isolating Non-Bulbar Dermal Sheath tissue; and

(d) cultivating the isolated Non-Bulbar Dermal sheath tissue to produce NBDS cells.

2) The method according to embodiment 1 wherein said vital hair is obtained by biopsy from a hair on the scalp of a subject.

3) The method according to any one of embodiments 1 or 2 wherein isolated NBDS cells can either be used autologous or allogeneic.

4) The method according to any one of embodiments 1 to 3 wherein said hair is cleaved utilizing a micromanipulator and scalpel, or scissors,

5) The method according to any one of embodiments 1 to 4, further comprising the step of conducting enzymatic digestion of said isolated Non-Bulbar Dermal Sheath tissue.

6) The method according to embodiment 5 wherein said enzymatic digestion is conducted with collagenase hyaluronidase, DNAse, elastase, papain, protease type XIV, trypsin, and dispase.

7) The method according to any one of embodiments 1 to 6 wherein said NBDS cells are passaged over multiple passages.

8) A composition comprising isolated Non-Bulbar Dermal Sheath cells prepared according to the method of any one of embodiments 1 to 7.

9) A composition comprising isolated Non-Bulbar Dermal Sheath cells.

10) A composition according to embodiments 8 or 9, further comprising serum plasma or platelet-rich plasma (PRP).

11) A composition according to any one of embodiments 8 to 10, further comprising fibrin and/or hyaluronic acid.

12) A composition according to any one of embodiments 8 to 11, further comprising components of the extracellular matrix, cytokines, chemokines and therapeutic agents.

13) The composition according to embodiment 12 wherein said components of the extracellular matrix are selected from the group consisting of glycosaminoglycans (GAGs), heparin sulfate, chondroitin sulfate, keratin sulfate, hyaluronic acid, elastin, collagens, fibronectins and laminins

14) The composition according to any one of embodiments 8 to 13, further comprising a scaffold.

15) The composition according to embodiment 14, wherein said scaffold is a biodegradable scaffold.

16) The composition according to embodiment 12 wherein said cytokines are selected from the group consisting of transforming growth factor beta (TGF-beta) and its isoforms, insulin-like growth factor (IGF) and its isoforms, granulocyte-macrophage colony-stimulating factor (GM-CSF), parathyroid-hormone-related protein, hepatocyte growth factor/scatter factor (HGF/SF), macrophage stimulating protein (MSP), epidermal growth factor (EGF), interleukin 6 (IL-6), stromal cell-derived factor 1 (SDF-1), platelet derived growth factor (PDGF) and fibroblast growth factor (FGF).

17) The composition according to embodiment 12 wherein said therapeutic agents are selected from the group consisting of analgesic agents, anti-inflammatory agents antibiotic, antiviral, antimycotic, and immunomodulatory agents.

18) A method for treating the skin, comprising the step of administering to the skin of a subject a composition according to any one of embodiments 8 to 17.

19) The method according to embodiment 18 wherein said subject is a mammal selected from the group consisting of humans, horse, dogs and cats.

20) The method according to embodiments 18 or 19 wherein said treatment is due to a skin injury

21) The method according to embodiment 20 wherein said skin injury is an acute or chronic wound or a scar.

22) The method according to embodiment 18 wherein said skin injury is a traumatic wound incurred by surgery, a burn, radiation, or accident.

23) The method according to embodiment 18 wherein said skin injury is a predisposition of spontaneous or induced bruises on the extremities.

24) The method according to embodiment 18 wherein said skin is aged skin.

25) The method according to embodiment 24 wherein said aged skin suffers from a condition selected from the group consisting of fine or coarse wrinkles, decreased skin thickness, decrease elasticity and firmness.

26) The method according to embodiment 18 wherein said skin is a subportion of the skin on a human subject selected from the group consisting of, the face, neck and hands.

27) A composition comprising NBDS cell supernatant.

28) A composition comprising NBDS cell supernatant made from the NBDS cells cultured according to the method of any one of embodiments 1 to 7.

29) The composition according to embodiments 27 or 28, wherein said composition is diluted or concentrated.

30) The composition according to any one of embodiments 27 to 29, further comprising an excipient.

31) The composition according to embodiment 30 wherein said excipient is a polymer.

32) The composition according to embodiment 30 formed into a paste, creme or gel.

33) Use of a composition according to any one of embodiments 27 to 32, for treatment of skin.

34) A method for treating skin, comprising administering to the skin of a subject a composition according to any one of embodiments 27 to 32.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-34. (canceled)

35. A method for treating or preventing skin aging or skin injury, the method comprising administering an effective amount of a composition to a skin of a subject in need thereof, the composition comprising isolated Non-Bulbar Dermal Sheath (NBDS) cells.

36. The method of claim 35 wherein the isolated NBDS cells are prepared by a method comprising:
(a) cleaving vital hair above a bulbar portion of a hair root to exclude a dermal papillae and a dermal sheath cup cell of the vital hair, but below a base of a sebaceous gland canal of the vital hair, to provide NBDS tissue isolated from the bulbar portion of the hair root;
(b) cultivating the isolated NBDS tissue to produce NBDS cells; and
(c) isolating the NBDS cells.

37. The method of claim 36 wherein the cultivating comprises passaging NBDS cells over multiple passages in serum-free media.

38. The method of claim 36 wherein the cultivating comprises passaging NBDS cells over at least four passages.

39. The method of claim 36 wherein the cultivating comprises culturing NBDS cells under conditions of low oxygen tension.

40. The method of claim 36 further comprising sorting the isolated NBDS cells to obtain a population of isolated NBDS cells.

41. The method of claim 36 further comprising sorting the isolated NBDS cells to obtain a population of isolated NBDS cells, wherein the sorting is by flow cytometry.

42. The method of claim 35 wherein the isolated NBDS cells of the composition are at least 95% pure, and the composition further comprises at least one contaminating cell type.

43. The method of claim 35 wherein the composition further comprises keratinocytes.

44. The method of claim 35 wherein the composition further comprises NBDS cell supernatant.

45. The method of claim 35 wherein the composition further comprises serum plasma or platelet-rich plasma (PRP).

46. The method of claim 35 wherein the composition further comprises fibrin and/or hyaluronic acid.

47. The method of claim 35 wherein the isolated NBDS cells are autologous.

48. The method of claim 35 wherein the isolated NBDS cells are allogeneic.

49. The method of claim 35 wherein the composition is in a form selected from a paste, crème or gel.

50. The method of claim 35 wherein the vital hair is taken from ultraviolet naïve tissue of the subject.

51. The method of claim 35 wherein the skin of the subject is injured skin.

52. The method of claim 35 wherein the skin injury is a traumatic wound incurred by surgery, a burn, radiation, or accident.

53. The method of claim 35 wherein the skin of the subject is aged skin.
